(19) United States
(12) Patent Application Publication
(10) Pub. No.: US 2025/0057974 A1
(43) Pub. Date: Feb. 20, 2025

(54) METHOD FOR CHANGING HYDROGEL VOLUME AND HYDROGEL

(71) Applicants: TOPPAN Holdings Inc., Taito-ku, Tokyo (JP); OSAKA UNIVERSITY, Suita-shi, Osaka (JP)

(72) Inventors: Michiya MATSUSAKI, Suita-shi, Osaka (JP); Masahiko NAKAMOTO, Suita-shi, Osaka (JP); Shiro KITANO, Taito-ku, Tokyo (JP)

(73) Assignees: TOPPAN Holdings Inc., Taito-ku, Tokyo (JP); OSAKA UNIVERSITY, Suita-shi, Osaka (JP)

(21) Appl. No.: 18/723,980

(22) PCT Filed: Dec. 14, 2022
(86) PCT No.: PCT/JP2022/046100

§ 371 Date: Jun. 25, 2024

(30) Foreign Application Priority Data
Dec. 28, 2021 (JP) 2021-213677

(51) Int. Cl. A61K 47/69; A61K 9/06; A61K 47/62; C08J 3/24; C08L 89/00

(52) U.S. Cl. CPC A61K 47/6903; A61K 9/06; A61K 47/62; C08J 3/246; C08L 89/00

(57) ABSTRACT

The present invention relates to a method for changing a hydrogel volume, the method including a step A of bringing an ionic polymer into contact with a hydrogel, which has a site having decomposition activity for the ionic polymer and a cross-linkable functional group that is capable of cross-linking with the ionic polymer through electrostatic interaction, to allow the cross-linkable functional group to cross-link with the ionic polymer, and reducing a volume of the hydrogel; and a step B of decomposing the ionic polymer cross-linking with the hydrogel by using the site having decomposition activity and discharging at least part of a decomposition product of the ionic polymer from the hydrogel to increase the hydrogel volume.

### TECHNICAL FIELD

The present invention relates to a method for changing a hydrogel volume and a hydrogel.

### BACKGROUND ART

In regard to artificial materials that respond to stimuli such as

biomolecules, for example, Non-Patent Literature 1 discloses a hydrogel that sustainedly releases a drug in response to an enzyme, and Non-Patent Literature 2 discloses a hydrogel scaffold that changes the structure (density and hardness) in response to an enzyme.

### CITATION LIST

### Patent Literature

### Non-Patent Literature

[Non-Patent Literature 1] Purcell, B. P.; Lobb, D.; Charati, M. B.; Dorsey, S. M.; Wade, R. J.; Zellars, K. N.; Doviak, H.; Pettaway, S.; Log-don, C. B.; Shuman, J. A.; Freels, P. D.; Gorman III, J. H.; Gorman, R. C.; Spinale, F. G.; Burdick, J. A., Nature Mater. 2014, 13, 653-661.

[Non-Patent Literature 2] Ooi, H. W.; Hafeez, S.; van Blitterswijk, C. A.; Moroni, L.; Baker, M. B., Hydrogels that listen to cells: a review of cell-responsive strategies in biomaterial design for tissue regeneration. Mater. Horiz. 2017, 4, 1020-1040.

### SUMMARY OF INVENTION

### Technical Problem

A Hydrogel (stimulus-responsive hydrogel) in which the volume changes in response to stimuli such as biomolecules can be applied to a drug sustained release system, tissue engineering, an actuator, and the like. However, in stimulus-responsive hydrogels in the related art, it is difficult to autonomously turn on and off functions in response to transient stimuli, or it is necessary to provide external stimuli respectively to turn on and off functions.

The present invention has been made in consideration of the above circumstances, and an object of the present invention is to provide a novel method for changing a hydrogel volume and a novel hydrogel.

### Solution to Problem

The present invention relates to, for example, each of the following inventions.
[1] A method for changing a hydrogel volume, the method comprising: a step A of bringing an ionic polymer into contact with a hydrogel, which has a site having decomposition activity for the ionic polymer and a cross-linkable functional group that is capable of cross-linking with the ionic polymer through electrostatic interaction, to allow the cross-linkable functional group to cross-link with the ionic polymer, and reducing a volume of the hydrogel; and
a step B of decomposing the ionic polymer cross-linking with the hydrogel by using the site having decomposition activity and discharging at least part of a decomposition product of the ionic polymer from the hydrogel to increase the volume of the hydrogel.
[2] The method according to [1], wherein the ionic polymer is an ionic biopolymer.
[3] The method according to [1] or [2], wherein the ionic polymer is a cationic polypeptide having a lysine residue.
[4] The method according to any one of [1] to [3], wherein a molecular weight of the ionic polymer is 530.70 or more.
[5] The method according to any one of [1] to [4], wherein the cross-linkable functional group is at least one selected from the group consisting of a carboxy group and a sulfate group.
[6] The method according to any one of [1] to [5], wherein the volume of the hydrogel is reduced by 10% to 80% by formation of a cross-linking structure with the ionic polymer.
[7] A hydrogel comprising:
a site having decomposition activity for an ionic polymer; and
a cross-linkable functional group that is capable of cross-linking with the ionic polymer through electrostatic interaction.
[8] The hydrogel according to [7], wherein the ionic polymer is a cationic polypeptide having a lysine residue.
[9] The hydrogel according to [7] or [8], wherein a molecular weight of the ionic polymer is 530.70 or more.
[10] The hydrogel according to any one of [7] to [9], wherein the cross-linkable functional group is at least one selected from the group consisting of a carboxy group and a sulfate group.
[11] The hydrogel according to any one of [7] to [10], wherein a volume of the hydrogel is reduced by 10% to 80% by formation of a cross-linking structure with the ionic polymer.

### Advantageous Effects of Invention

According to the present invention, it is possible to provide a novel method for changing a hydrogel volume and a novel hydrogel.

According to the method for changing a hydrogel volume according to the present invention, it is possible to spontaneously and dynamically increase or decrease the volume using an ionic polymer as a fuel.

### BRIEF DESCRIPTION OF DRAWINGS

FIG. 1 is a diagram for describing one embodiment of a method for changing a hydrogel volume.

FIG. 2 is a diagram that shows one example of a method for producing a hydrogel (hereinafter also referred to as “AT-gel”) containing a site where trypsin has been immobilized, and a carboxy group as a cross-linkable functional group.

FIG. 3 shows graphs showing results obtained by analyzing acrylated trypsin that is used in the production of the AT-gel, where (A) and (B) are graphs showing results obtained by MALDI-TOF-MS analysis of trypsin before acrylation (Native trypsin) and acrylated trypsin, and (C) is a graph showing results obtained by analyzing acrylated trypsin using fluorescamine.

FIG. 4 is a graph showing results obtained by measuring volume changes of the AT-gel in response to α-poly-L-lysine (hereinafter also referred to as “PL”)

FIG. 5 is a photographic image that shows results of the observation of the volume change of the AT-gel in response to PL.

FIG. 6 is a graph showing results obtained by measuring the volume change of the AT-gel in a case where PL addition (arrow in the figure) is repeated a plurality of times.

FIG. 7 is a graph showing results obtained by analyzing di-lysine and tri-lysine, which are present in the supernatant of the AT-gel.

FIG. 8 is a photographic image that shows the results of analyzing poly-lysine, di-lysine, and tri-lysine by thin-layer chromatography (TLC) in the process of the volume change.

FIG. 9 is a graph showing results obtained by measuring binding amounts of a hydrogel to PL or di-lysine, the hydrogel having been pretreated with an irreversible trypsin inhibitor (4-(2-aminoethyl) benzenesulfonyl fluoride (AEBSF)).

FIG. 10 is a graph showing results obtained by measuring the volume change of the AT-gel pretreated with AEBSF, in response to PL or di-lysine.

FIG. 11 is a graph showing results obtained by measuring the volume change of the AT-gel in response to di-lysine.

FIG. 12 is a schematic diagram for describing the inhibition of a transient volume change by using a trypsin inhibitor or a trypsin inhibitor.

FIG. 13 is a graph showing results obtained by measuring volume changes of the AT-gel in the presence or absence of a reversible trypsin inhibitor (4-aminobenzamidine (ABA)) or free acrylated trypsin.

FIG. 14 is a graph showing the volume change of the AT-gel at various PL concentrations.

FIG. 15 shows graphs showing the volume change of the AT-gel at various PL concentrations, where (A) is a graph showing results at 0.0 gL⁻¹, (B) is a graph showing results at 0.5 gL⁻¹, (C) is a graph showing results at 1.0 gL⁻¹, (D) is a graph showing results at 1.5 gL⁻¹, (E) is a graph showing results at 2.0 gL⁻¹, and (F) is a graph showing results at 2.5 gL⁻¹.

FIG. 16 is a graph showing results obtained by measuring the amplitude of the volume changes of the AT-gel.

FIG. 17 is a graph showing the time decay constant in each of the shrinkage and the re-swelling of the AT-gel.

FIG. 18 is a diagram showing a hypothetical mechanism of secretion of a supported substance (methylene blue (MB)) by the AT-gel.

FIG. 19 is a graph showing results obtained by measuring the release of MB by the AT-gel in response to the addition of 1 gL⁻¹ PL. The arrows in the figure indicate the time at which PL is added.

FIG. 20 is a graph showing results obtained by measuring the release of MB by the AT-gel in response to the addition of 1 gL⁻¹ PL. The arrows in the figure indicate the time at which PL is added.

### DESCRIPTION OF EMBODIMENTS

Hereinafter, embodiments for executing the present invention will be described in detail. However, the present invention is not limited to the following embodiments.

A method for changing a hydrogel volume according to the present embodiment includes a step A of bringing an ionic polymer into contact with a hydrogel, which has a site having decomposition activity for the ionic polymer and a cross-linkable functional group that is capable of cross-linking with the ionic polymer through electrostatic interaction, to allow the cross-linkable functional group to cross-link with the ionic polymer, and reducing a volume of the hydrogel, and a step B of decomposing a cross-linking structure formed by the ionic polymer by using the site having decomposition activity and discharging at least part of a decomposition product of the ionic polymer from the hydrogel to increase the volume of the hydrogel.

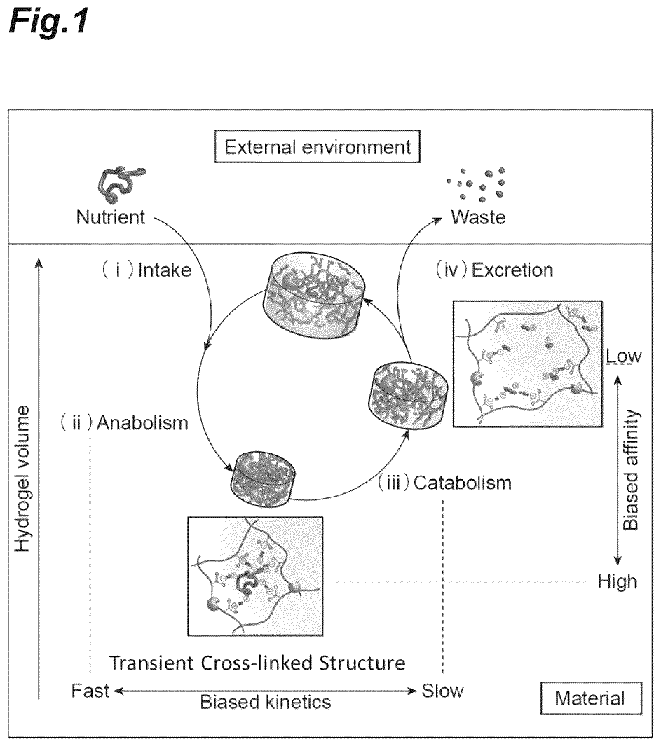

FIG. 1 is a diagram for describing one embodiment of a method for changing a hydrogel volume. In this method, as shown in FIG. 1, the hydrogel volume changes through a cycle including (i) intake, (ii) anabolism, (iii) catabolismand (iv) excretion. The vertical axis in FIG. 1 indicates the hydrogel volume. Hereinafter, the method for changing a hydrogel volume, which includes the step A and the step B, will be described with reference to FIG. 1 as necessary.

### Step A

Step A is a step of brining an ionic polymer into contact with a hydrogel to allow the cross-linkable functional group to cross-link with the ionic polymer, and reducing a volume of the hydrogel.

### Ionic Polymer

The ionic polymer is a polymer having an ionic functional group. The ionic functional group may be a cationic functional group or an anionic functional group. Examples of the cationic functional group include an amino group (—NH₂), an imidazolyl group, and a guanidino group. Examples of the anionic functional group include a carboxy group (—COOH) and a sulfate group (—SO₃H).

The ionic polymer may be an ionic biopolymer. The ionic biopolymer may be, for example, a polypeptide, a protein, a polynucleotide, or a polysaccharide.

For example, the ionic biopolymer may be an ionic polypeptide or may be a cationic polypeptide or an anionic polypeptide. The cationic polypeptide is a polypeptide containing basic amino acid residues. The basic amino acid residue may be, for example, a lysine residue, an arginine residue, or a histidine residue. The anionic polypeptide is a polypeptide containing acidic amino acid residues. The acidic amino acid residue may be, for example, aspartic acid or glutamic acid. The ionic polypeptide may be a cationic polypeptide having lysine residues.

The isoelectric point of the cationic polypeptide may be, for example, 10 to 12. The isoelectric point refers to a pH at which a charge average of an entire compound is 0 in a case where the compound is dissolved in water and ionized.

Specific examples of the ionic polymer include poly-lysine (for example, α-poly-L-lysine), polyarginine, lysozyme, and cytochrome C.

The molecular weight of the ionic polymer may be, for example, 530.70 or more, 1,000 or more, 5,000 or more, 10,000 or more, 20,000 or more, or 30,000 or more. The upper limit of the molecular weight of the ionic polymer is not particularly limited; however, it may be, for example, 1,000,000 or less, 500,000 or less, 100,000 or less, 80,000 or less, or 70,000 or less. The molecular weight of the ionic polymer is the sum of the atomic weights of the constituent atoms of the ionic polymer, and in a case where the ionic polymer is a polypeptide, the molecular weight of the ionic polymer is the sum of the molecular weights of all amino acid residues that constitute the ionic polymer.

The viscosity average molecular weight (viscometric Mw) of the ionic polymer may be, for example, 10,000 or more, 20,000 or more, or 30,000 or more. The upper limit of the viscosity average molecular weight of the ionic polymer is not particularly however, it may be, for example, 100,000 or less, 80,000 or less, or 60,000 or less.

The number of amino acid residues of the ionic polypeptide may be 4 or more, 10 or more, 50 or more, 100 or more, 150 or more, 200 or more, 250 or more, 300 or more, or 350 or more. The upper limit of the number of amino acid residues of the ionic polypeptide is not particularly limited; however, it may be, for example, 1000 or less, 800 or less, 600 or less, 550 or less, 500 or less, 450 or less, or 400 or less.

### Hydrogel

The hydrogel contains a polymer and water. The hydrogel has a three-dimensional network structure that is formed by a polymer, and water is contained in the inside of the three-dimensional network structure. The hydrogel can also contain a substance other than water.

The hydrogel according to the present embodiment includes a site having decomposition activity for an ionic polymer; and a cross-linkable functional group that is capable of cross-linking with the ionic polymer through electrostatic interaction.

The cross-linkable functional group is a functional group that is capable of cross-linking with an ionic functional group in an ionic polymer through electrostatic interaction. The cross-linkable functional group may be the cationic functional group or the anionic functional group. The cross-linkable functional group may contain at least one selected from the group consisting of a carboxy group and a sulfate group.

The structure of the site having decomposition activity for an ionic polymer can be appropriately designed according to the kind of ionic polymer, the kind of cross-linkable functional group, and the like. For example, the site having decomposition activity for an ionic polymer may be a site where an enzyme has been immobilized (enzyme immobilized site). The enzyme may be a protease, a glycosidase, or a nuclease. In a case where the ionic polymer is a polyamino acid (for example, a polypeptide or a protein), a protease can be used as an enzyme. In a case where the ionic polymer is a polysaccharide, a glycosidase can be used as an enzyme. In a case where the ionic polymer is a polynucleotide (DNA, RNA, or the like), a nuclease can be used as an enzyme. Examples of the enzyme include trypsin, chymotrypsin, pepsin, a matrix metalloproteinase, amylase, lysozyme, a deoxyribonuclease, and a ribonuclease.

The polymer that constitutes the hydrogel is formed through a polymerization reaction of polymerizable monomers. The polymerizable monomer has a polymerizable functional group. The number of polymerizable functional groups per one polymerizable monomer molecule may be, for example, 1 to 7, and may be 1 or 2.

Examples of the polymerizable functional group include a group having an ethylenically unsaturated bond. Examples of the group having an ethylenically unsaturated bond include an acryloyl group, a methacryloyl group, an acrylamide group, a methacrylamide group, and a vinyl group.

The polymer that constitutes the hydrogel may contain, as a monomer unit, a polymerizable monomer having a cross-linkable functional group. Examples of the polymerizable monomer having a cross-linkable functional group include acrylic acid, methacrylic acid, vinyl sulfate, and 2-acrylamide-2-methyl-1-propanesulfonic acid.

In the polymer network, the calculated density as a monomer unit of a polymerizable monomer having a cross-linkable functional group may be, for example, 1 to 50 mM, 5 to 30 mM, or 10 to 25 mM. In the present specification, the calculated density in the polymer network can be calculated using the following expression.

Calculated density=ρ×X/M
ρ (g/L): A density of a hydrogel hydrated in a buffer solution, which is determined from the dry weight in pure water and the degree of swelling (in buffer solution/in pure water)
X (g/g): Preparation ratio
M (g/mol): Molecular weight of each monomer

The preparation ratio (X) is calculated according to the expression: each monomer amount (g)/total monomer amount (g).

The polymer that constitutes the hydrogel may contain, as a monomer unit, a polymerizable monomer to which a substance having decomposition activity for an ionic polymer has been bound. A polymerizable monomer to which a substance having decomposition activity for an ionic polymer has been bound may be, for example, an enzyme into which a polymerizable functional group has been introduced, and it may be, for example, acrylated trypsin.

In the polymer network, the calculated density as a monomer unit of a polymerizable monomer to which a substance having decomposition activity for an ionic polymer is bound may be 0.1 to 10 μM or 0.5 to 5 μM.

The polymer that constitutes the hydrogel may contain a monomer (another polymerizable monomer) that does not correspond to the polymerizable monomer. The polymer that constitutes the hydrogel may contain, as another polymerizable monomer, a monofunctional polymerizable monomer having one polymerizable functional group. Examples of the monofunctional polymerizable monomer include acrylamide, N-isopropylacrylamide, methacrylamide, N-isopropyl methacrylamide, and 2-hydroxypropyl methacrylamide.

The polymer that constitutes the hydrogel may contain, as another polymerizable monomer, a polyfunctional polymerizable monomer having two or more polymerizable functional groups.

Examples of the polyfunctional polymerizable monomer include N,N′-methylenebisacrylamide, N,N′-ethylenebisacrylamide, polyethylene glycol diacrylate, N,N′-methylenebismethacrylamide, N,N′-ethylenebismethacrylamide, and polyethylene glycol dimethacrylate.

The polymer that constitutes the hydrogel may include a polymerizable monomer to which a fluorescent dye has been bound.

The polymer concentration in the hydrogel may be, for example, 1 to 10 g/L, 3 to 8 g/L, or 5 to 7 g/L based on the total volume of the hydrogel.

The hydrogel according to the present embodiment can be produced by a method that includes polymerizing a polymerizable monomer in a reaction solution containing the polymerizable monomer and water. The conditions for polymerization can be appropriately set according to the kind, amount, and the like of the polymerizable monomer.

The pH of the reaction solution is not particularly limited; however, it may be adjusted to, for example, 2.5 to 3.4. The pH can be adjusted using an alkaline aqueous solution such as an aqueous sodium hydroxide solution or an acidic aqueous solution such as an aqueous hydrogen chloride solution.

A polymerization initiator may be used for the polymerization reaction. The polymerization initiator can be appropriately selected according to the kind of polymerizable monomer. Examples of the polymerization initiators include ammonium persulfate (APS) and N,N,N′,N′-tetramethylethylenediamine (TEMED).

The reaction solution may contain calcium chloride (CaCl₂). In this case, autodecomposition and/or denaturation during polymerization is more easily suppressed.

The temperature of the reaction solution at the time of reaction may be, for example, 20°° C. to 30° C. For example, the time for holding the temperature of the reaction solution in the above-described range may be 5 minutes or more and may be 1 hour or less.

### Intake and Catabolism

In the step A, as shown in FIG. 1, (i) intake and (ii) anabolism are carried out. The hydrogel volume is reduced through (i) intake and (ii) anabolism as shown in FIG. 1.

### (i) Intake

As shown in FIG. 1, an ionic polymer supplied from the external environment is brought into contact with a hydrogel, whereby the ionic polymer is incorporated into the hydrogel through electrostatic interaction. A method of bringing an ionic polymer into contact with a hydrogel is not particularly limited, and examples thereof include a method of immersing a hydrogel in a solution containing an ionic polymer and a method of adding an ionic polymer to a hydrogel.

### (ii) Anabolism

The cross-linkable functional group in the hydrogel is cross-linked with the ionic polymer incorporated into the hydrogel. That is, the ionic polymer incorporated into the hydrogel is used as a building block for forming a cross-linking structure in the hydrogel. The cross-linking structure is formed through electrostatic interaction. As shown in FIG. 1, in a case where the cross-linkable functional group is a carboxy group, and the ionic polymer is a cationic polypeptide, the cross-linking structure is formed through the interaction between the negative charge in the carboxylate ion and the positive charge in the cationic polypeptide.

Due to the formation of the cross-linking structure, the hydrogel volume decreases as compared with the hydrogel volume before the formation of the cross-linking structure. The hydrogel volume may be reduced by 10% to 80% by the formation of a cross-linking structure with an ionic polymer. The rate of reduction in the hydrogel volume due to the formation of a cross-linking structure with an ionic polymer may be, for example, 20% to 70% or 30% to 60%. The rate of reduction in the hydrogel volume due to the formation of a cross-linking structure with an ionic polymer can be measured according to the method described in Examples described below.

Since at least a part of the cross-linking structure formed through anabolism is decomposed through (iii) catabolismand (iv) excretion, it can also be said that the cross-linking structure can also be referred to as a transient cross-linked structure.

### Step B

The step B is a step of decomposing the ionic polymer cross-linking with the hydrogel by using the site having decomposition activity and discharging at least part of a decomposition product of the ionic polymer from the hydrogel to increase the hydrogel volume.

### Catabolismand Excretion

In the step B, as shown in FIG. 1, (iii) catabolismand (iv) excretion are carried out. The hydrogel volume increases through (iii) catabolismand (iv) excretion, which are shown in FIG. 1.

(iii) Catabolism

The site having the decomposition activity for the ionic polymer in the hydrogel decomposes at least a part of the cross-linking structure formed through the electrostatic interaction between the cross-linkable functional group in the hydrogel and the ionic polymer. The ionic polymer is oligomerized by decomposition.

### (iv) Excretion

The oligomerized ionic polymer is released from the hydrogel. Since the cross-linking structure is decomposed, and the decomposition product (oligomerized product) of the ionic polymer is released from the hydrogel, the hydrogel volume increases as compared with the hydrogel volume in which the cross-linking structure is formed.

The hydrogel volume after the step A and the step B may be, for example, 70% to 100%, 80% to 100%, or 90% to 100% of the hydrogel volume before the step A.

The step A and the step B may be carried out repeatedly. For example, in the step B, the hydrogel volume is increased, and then an ionic polymer is supplied to bringing the ionic polymer into contact with the hydrogel again, whereby the hydrogel volume can be reduced again.

As shown in FIG. 1, the affinity of the hydrogel for an ionic polymer before oligomerization is high as compared with the affinity of the hydrogel for an oligomerized product (waste) of the ionic polymer. It is presumed that such a biased affinity occurs since the ionic polymer before oligomerization exhibits a larger influence on the stability of the poly-ion complex. That is, it is presumed that (i) intake and (iv) excretion are driven by the biased affinity of the ionic polymer between before oligomerization and after oligomerization. As shown in FIG. 1, the construction rate of the cross-linking structure (electrostatic cross-links) through electrostatic interaction is low as compared with the decomposition rate of the electrostatic cross-link. It is presumed that such a difference in rate occurs since the movement of the site (for example, a site where an enzyme has been immobilized) having decomposition activity is greatly limited as compared with a free enzyme in a bulk solution. That is, it is presumed that (ii) anabolism and (iii) catabolism are driven by the biased kinetics between the construction rate of electrostatic cross-links and the decomposition rate of electrostatic cross-links.

The hydrogel according to the present embodiment and the method for changing the hydrogel volume can be used for a drug sustained release system, tissue engineering (for example, a scaffolding material in tissue engineering), an actuator, and the like.

As one embodiment of the present invention, a formulation containing a drug and the above-described hydrogel that retains the drug is provided. In the formulation, the release of the drug retained in the hydrogel can be controlled by changing the hydrogel volume. The formulation can be also referred to as a controlled release formulation. The formulation can also be used as a sustained release formulation.

As one embodiment of the present invention, a method for controlling release of a drug or a method for sustainedly releasing a drug is provided. The method includes a step of brining an ionic polymer into contact with the hydrogel retaining a drug to allow the cross-linkable functional group in the hydrogel to cross-link with the ionic polymer, and reducing a volume of the hydrogel, and releasing the drug retained in the hydrogel; and a step of decomposing the ionic polymer cross-linking with the hydrogel by using the site having decomposition activity in the hydrogel and discharging at least part of a decomposition product of the ionic polymer from the hydrogel to increase the hydrogel volume. The above-described aspects can be applied to the method for reducing the hydrogel volume and the method for increasing the hydrogel volume.

### EXAMPLES

Hereinafter, the present invention will be more specifically described based on Examples. However, the present invention is not limited to Examples below.

### Materials and Equipment Used

Regarding the following substances, commercially available products were used. Acrylamide, acrylic acid, N,N′-methylenebisacrylamide, ammonium persulfate (APS), and acryloyl chloride were obtained from Tokyo Chemical Industry Co., Ltd. (Tokyo, Japan). N,N,N′, N′-tetramethylethylenediamine (TEMED), trypsin, di-lysine, and tri-lysine were obtained from Sigma-Aldrich Co., LLC (St. Louis, Missouri, USA). α-poly-L-lysine hydrobromide (molecular weight: 30 to 70 kDa (viscometric Mw: 47 kDa)) was obtained from Nacalai Tesque Inc. (Kyoto, Japan). L-lysine, sodium bicarbonate, and 10 N sodium hydroxide were obtained from FUJIFILM Wako Pure Chemical Corporation (Osaka, Japan). A dialysis tube (MWCO: 3,500 Da) was obtained from Fisher Bland, Inc. and was washed with MilliQ before use. A proton nuclear resonance (¹H NMR) spectrum was measured in d6-DMSO at room temperature with a JNM-ECS400 (JEOL Ltd., Tokyo, Japan) spectrometer. A fluorescence spectrum was measured using FP-8500 (Jasco Ltd., Tokyo, Japan). ESI-MS analysis was carried out using JMS-T100LP (JEOL Ltd., Tokyo, Japan). MALDI-TOF-MS analysis was carried out using Autoflex III (Bruker Biospin Daltonics, Bremen, Germany). Lyophilization of acrylated trypsin (AcTryp) was carried out using FDU-2200 (EYLA).

### Production of Hydrogel

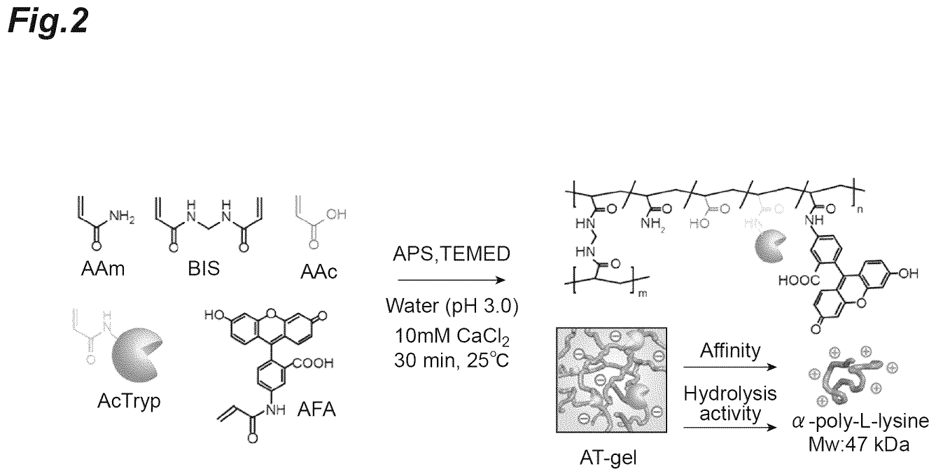

A hydrogel having an affinity for α-poly-L-lysine (PL) and hydrolytic activity (hereinafter, also referred to as an “AT-gel”) was prepared by a free radical copolymerization of acrylamide (AAm, 200 gL-1), N,N′-methylenebisacrylamide (BIS, 0.5 gL⁻¹), acrylic acid (AAc, 50 gL⁻¹), and acrylated trypsin (AcTryp, 1 gL⁻¹) (FIG. 2). A small amount of an acrylamide fluorescent dye (AFA, 0.25 gL⁻¹) was also introduced into the hydrogel (AT-gel) for visualization.

### Synthesis of AcTryp

Polymerizable trypsin (AcTryp) was synthesized through the bonding between an N-hydroxysuccinimide ester activating compound and a lysine residue on trypsin according to a method obtained by slightly modifying the method described in the known document (Yasayan, G. et. al., Polym. Chem. 2011, 2, 1567-1578.). Trypsin (200 mg, 0.009 mmol) and benzamidine dihydrochloride (13 mg, 0.08 mmol) were dissolved in 20 mL of a 100 mM phosphate buffer solution (pH: 7.5). N-hydroxysuccinimide acrylate (37 mg, 0.22 mmol) was dissolved in 1 mL of DMSO, added dropwise to the solution, and incubated at 25° C. for 90 minutes. The obtained reaction solution was dialyzed at 4° C. for one day against a 1 mM HCl aqueous solution containing 10 mM CaCl2 and for one day against MilliQ (MWCO: 3,500 Da). The obtained solution was filtered (pore diameter: 0.4 μm) and lyophilized to obtain a white powder (yield: 72%).

### Analysis of AcTryp

### Analysis of AcTryp by MALDI-TOF-MS

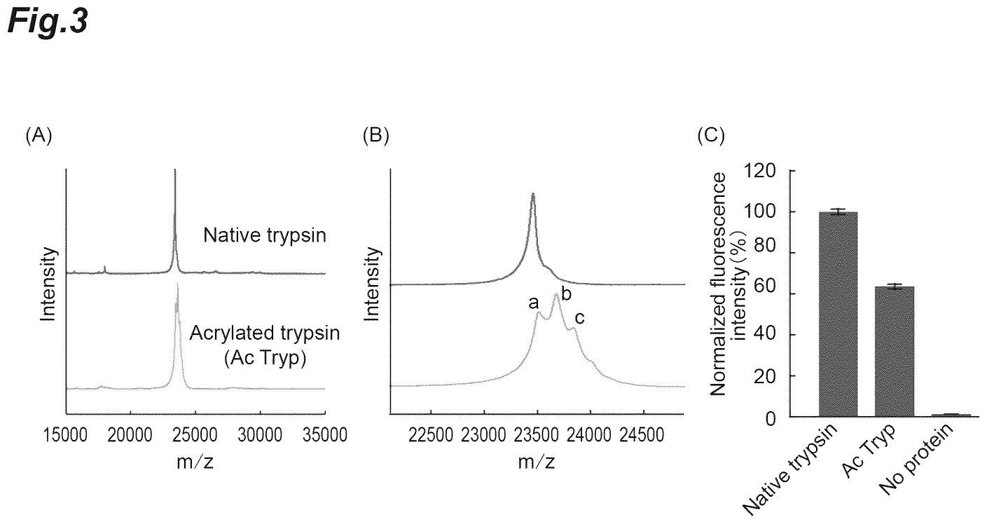

The number of conjugated acrylate groups on trypsin was investigated by MALDI-TOF-MS using the method reported in the known document (Yasayan, G. et. al., Polym. Chem. 2011, 2, 1567-1578.) An aqueous solution of the conjugate (1.0 gL⁻¹) was mixed with an equal amount of a matrix (8 mg of sinapic acid (50/50 (v/v)) in 1 mL of water/MeCN). 2 μL of the mixture was spotted onto a plate target and dried. The number of conjugated acrylate groups was determined by comparing the molecular weight of conjugated trypsin with the molecular weight of natural trypsin. After being bound, acrylated trypsin (AcTryp) showed an increased molecular weight as compared with natural trypsin (FIG. 3(A) and FIG. 3(B)). Three peaks of a, b, and c of AcTryp showed mass increases of 54.6 (m/z), 221.3 (m/z), and 382.9 (m/z), respectively. These correspond to the conjugation of 1,4, and 7 acrylates per one molecule of trypsin (the theoretical m/z value of one acrylate is 54.01).

### Analysis of AcTryp With Fluorescamine

The number of conjugated acrylate groups was also evaluated from the amount of residual primary amines of the trypsin stained with fluorescamine (FIG. 3 (C)). 0.1 gL⁻¹ of natural trypsin or 0.1 gL⁻¹ of AcTryp in 10 mM NaHCO₃ (pH: 8.5) was mixed with a 10 vol % fluorescamine solution (0.5 gL⁻¹ in DMSO) at room temperature. Incubation for 15 minutes was followed. The fluorescence intensity of the mixture was measured with a fluorescence spectrometer (excitation wavelength: 395 nm, fluorescence wavelength: 495 nm). The strength of AcTryp is 64% as compared with that of natural trypsin, which indicates the average binding of five acrylates on a single trypsin. This is consistent with the results of the mass spectrometry analysis.

### Synthesis of AFA

Acrylamide (AFA) conjugated with a fluorescent dye was synthesized in accordance with the previous report (Serpe, M. J.; Jones, C. D.; Lyon, L. A., Langmuir 2003, 19, 8759-8764.). 4-aminofluorescein (125 mg, 0.36 mmol) was suspended in 20 ml of dry acetone, and acryloyl chloride (32 μL, 0.39 mmol) was added dropwise to a 0° C. solution. While stirring, the solution was reacted at room temperature for 3 hours. Yellow crystals were recovered by filtration and washed with cold acetone and diethyl ether. The product was further purified by recrystallization from anhydrous THF and dried overnight under vacuum (yield=43%).
¹H NMR (400 MHZ, DMSO-d6, δ): 5.81 (2d,1H), 6.33 (2d, 1H), 6.4-6.7 (m, 1H), 7.21 (d, 1H), 8.39 (d, 1H), 10.8 (s, 1H).

### Synthesis of Hydrogel

A hydrogel having a polymer concentration of 6.3 gL⁻¹ at equilibrium was obtained by the method shown below. The calculated densities of the AAc unit and the AcTryp unit in the polymer network were 17 mM and 1 μM, respectively.

AAm, BIS, AAc, and AFA were dissolved in MilliQ containing 10 mM CaCl₂ to prepare a monomer solution. CaCl₂ was used to avoid self-dissolution and/or denaturation of trypsin during polymerization (Sipos, T. et al., Biochemistry, 1970, 9, 2766-2775.).

The pH of the monomer solution was adjusted to about 3 by adding 10 N NaOH, and then AcTryp was added to the solution. The monomer solution was degassed by bubbling nitrogen on ice for 10 minutes. APS and TEMED were added to the monomer solution as polymerization initiators (the final concentration of both APS and TEMED was set to 5 gL⁻¹). Immediately after the addition of the polymerization initiator, the monomer solution was transferred to a container assembled from a glass plate and a silicon spacer, and polymerization was carried out at 25° C. for 30 minutes. The hydrogel prepared in this way, which had a width of 40 mm, a thickness of 0.1 mm, and a length of 40 mm, was sequentially washed with MilliQ and pre-incubated at 4° C. for one day in a 10 mM NaHCO3 buffer solution (pH: 8.5). As a result, a hydrogel having a polymer concentration of 6.3 gL⁻¹ at equilibrium was obtained. The polymer density of the hydrogel in MilliQ was obtained from the weights before and after lyophilization. The polymer density of the hydrogel in a 10 mM NaHCO₃ buffer solution (pH: 8.5) was obtained from the swelling ratio of the hydrogel, which was obtained by comparing the swelling with that in MilliQ.

Table 1 shows the amounts of monomers used in the synthesis of the AT-gel, the A-gel, and the T-gel. The A-T-gel is a hydrogel containing a polymer that contains a site in which trypsin is immobilized and contains a carboxy group as a cross-linkable functional group. The A-gel and the T-gel are a hydrogel that does not contain a site in which trypsin is immobilized, or a carboxy group as a cross-linkable functional group.

TABLE 1

| | AAm | BIS | AAc | AcTrp | AFA |
|---|---|---|---|---|---|
| | (gL⁻¹) | (gL⁻¹) | (gL⁻¹) | (gL⁻¹) | (gL⁻¹) |
| AT-gel | 200 | 0.5 | 100 | 1 | 0.25 |
| A-gel | 200 | 0.5 | 100 | 0 | 0.25 |
| T-gel | 200 | 0.5 | 0 | 1 | 0.25 |

### Evaluation of Transient Volume Change of Hydrogel

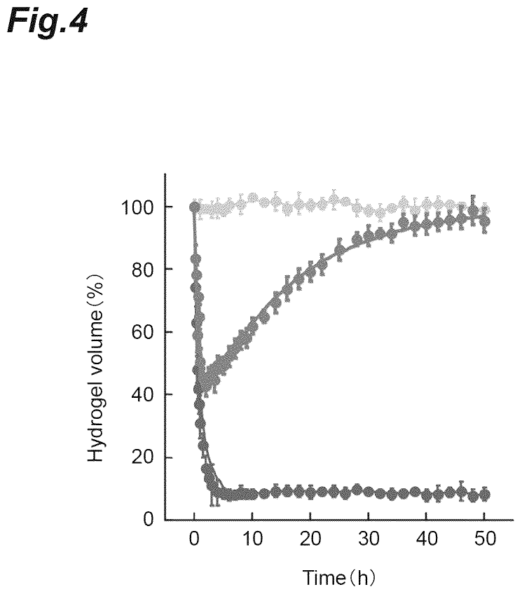

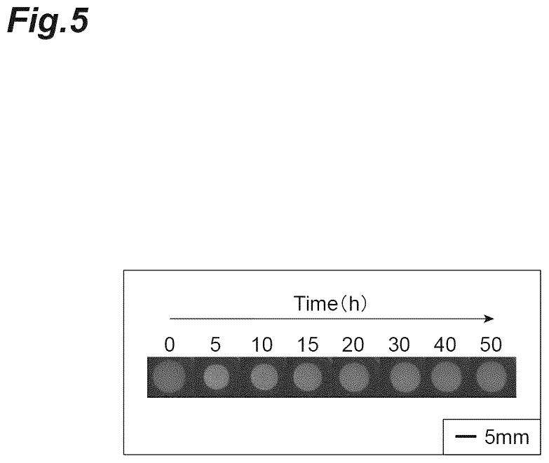

The hydrogel was cut into a disc shape (diameter: 8 mm, thickness: 0.4 mm, polymer weight: 0.5 mg) and immersed in 1 mL of 10 mM NaHCO₃ containing 2 gL⁻¹ PL at 25° C., and the volume change of the hydrogel was investigated as a function of time (FIG. 4 and FIG. 5). The volume change of the hydrogel was determined by the ratio of the diameter change measured by ImageJ, which was obtained by assuming isotropic shrinkage/expansion (Miyata, T.; Asami, N.; Uragami, T., A reversibly antigen-responsive hydrogel. Nature 1999, 399, 766-769.).

FIG. 4 shows the volume change of the AT-gel, the A-gel, and the T-gel in response to the PL addition. In FIG. 4, the dark color plot shows the results in a case where the A-gel has been used, the light color plot shows the results in a case where the T-gel has been used, and the intermediate color plot shows the results in a case where the AT-gel has been used. FIG. 5 is a photographic image showing an example of the volume change of the AT-gel under ultraviolet light (365 nm).

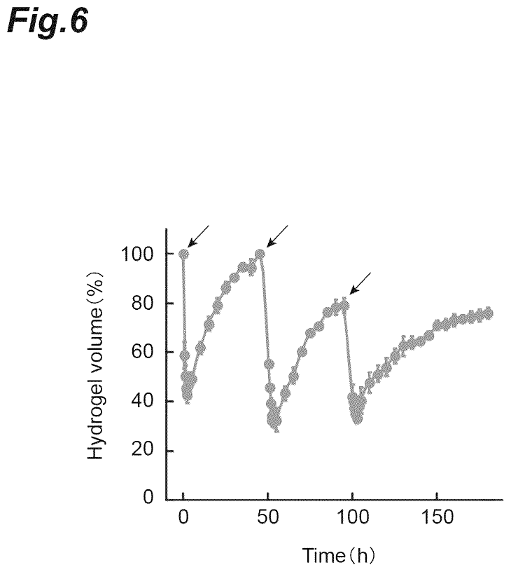

The volume change of the AT-gel was macroscopic and transient. The volume of the AT-T gel reduced to 43%+6.3% within 2 hours, and in the equilibrium state, it increased autonomously and slowly to 94%+3.8% as compared with the initial state. The shrinkage of the A-gel was observed, but the re-swelling thereof was not observed. The volume change of the T-gel was not observed. These results indicate that the AT-gel captures PL through electrostatic interaction and shrinks by the construction of electrostatic cross-links (see the following documents a to c). It is considered that the autonomous re-swelling of the AT-gel results from the hydrolysis of the cross-linking structure by trypsin. The substitution of the supernatant with a new solution containing PL (2 gL⁻¹) could make the transient volume change of the AT-gel extend by at least three additional cycles of volume change (FIG. 6). Although the increase in the number of cycles did not affect the shrinkage, it slowed down the re-swelling process. Conceivably, denaturation and/or autolysis of trypsin is a factor that slows down the re-swelling process. From these results, the inventors of the present invention concluded that the combination of the two kinds of functions provided by AAc and AcTryp and the affinity and hydrolytic activity for PL contributes to the transient volume change of the AT-gel.
(a) Smith, M. H.; Lyon, A. L., Tunable Encapsulation of Proteins within Charged Microgels. Macromolecules 2011, 44, 8154-8160.
(b) Sun, T. L.; Kurokawa, T.; Kuroda, S.; Ihsan, A. B.; Akasaki, T.; Sato, K.; Haque, M. A.; Nakajima, T.; Gong, J. P., Physical hydrogels composed of polyampholytes demonstrate high toughness and viscoelasticity. Nat. Mater. 2013, 12, 932-937.
(c) Mansson, R.; Frenning, G.; Malmsten, M., Factors Affecting Enzymatic Degradation of Microgel-Bound Peptides. Biomacromolecules 2013, 14, 7, 2317-2325.

### Contribution of Biased Affinity Between Nutrient and Waste to Transient Volume Change of Hydrogel

Further evaluation of the factors of the transient volume change of the AT-gel was carried out to understand the mechanism. (Analysis of transient volume change by ESI-MS)

The AT-gel that had been cut into a circle (diameter: 8 mm, thickness: 0.4 mm, polymer weight: 0.5 mg) was immersed at 25° C. for 50 hours in 1 mL of 10 mM NaHCO₃ containing 2 gL⁻¹ PL. After the transient volume change of the AT-gel, the supernatant was recovered and diluted 100-fold with methanol. Peaks arising from hydrolyzed oligo-lysine (di-lysine and tri-lysine) were observed by positive mode electrospray ionization mass spectrometry.

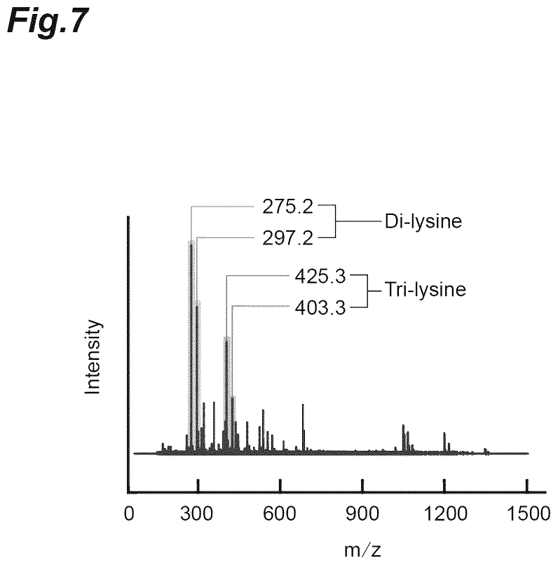

The di-lysine and tri-lysine in the supernatant after the transient volume change of the AT-gel were identified by electrospray ionization mass spectrometry (ESI-MS) (FIG. 7). These results are consistent with the previous report on the action of trypsin on PL (Waley, S. G. et al., Biochem. J. 1953, 55, 328-337.).

### Analysis of Transient Volume Changes by Thin-Layer Chromatography

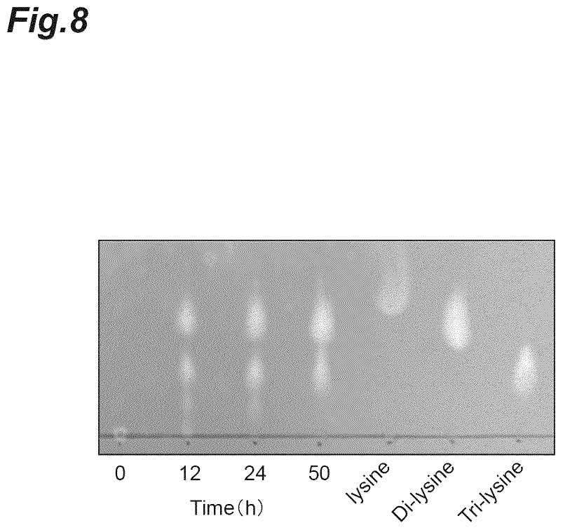

The AT-gel that had been cut into a circle (diameter: 8 mm, thickness: 0.4 mm, polymer weight: 0.5 mg) was immersed in 10 mL of 1 mM NaHCO₃ containing 2 gL⁻¹ PL at 25° C. The supernatant was analyzed by thin-layer chromatography (TLC, Waley, S. G. et al., Biochem. J. 1953, 55, 328-337.) at various points (n-butanol: acetic acid: water: pyridine=30:6:24:20). The TLC plate was stained by spraying it with fluorescamine (0.05% in acetone), and the image was captured under UV light (λ=365 nm). It was confirmed that at the bottom, the spot from the full-length PL decreases gradually, and a spot emerges from the oligo-lysine (FIG. 8). After 50 hours, the spot from the full-length PL completely disappeared, and di-and tri-lysine were observed.

Thin-layer chromatography analysis of the supernatant revealed that in the course of the transient volume change, the supplied full-length PL reduced, and the oligo-lysine increased. At equilibrium, the results were such that PL was completely converted to di-lysine or tri-lysine (FIG. 8).

### Analysis of Boding of PL and Di-Lysine to AT-Gel

The binding affinity of the AT-gel for the full-length PL and the di-lysine was investigated. To inhibit the hydrolysis of PL during the binding assay, the AT-gel was pretreated with 5 mM 4-(2-aminoethyl) benzenesulfonyl fluoride (AEBSF), which is an irreversible trypsin inhibitor.

The AT-gel that had been cut into a circle (diameter: 8 mm, thickness: 0.4 mm, polymer weight: 0.5 mg) was immersed in 1 mL of 5 mM [4-(2-aminoethyl) benzenesulfonyl fluoride (AEBSF) and incubated at 4° C. for 15 hours. The hydrogel was immersed in 10 mL of 1 mM HCl at room temperature. It was treated for 15 hours to remove unreacted AEBSF. The solution was substituted three times, followed by immersion in MilliQ at room temperature. The hydrogel was immersed in 1mL of 10 mM NaHCO₃, which contained PL in various concentrations or di-lysine (0.0 gL⁻¹, 0.5 gL⁻¹, 1.0 gL⁻¹, 1.5 gL⁻¹, 2.0 gL⁻¹, and 2.5 gL⁻¹), and the solution was incubated at 25° C. for 24 hours.

The concentration of PL or di-lysine in the supernatant was quantified by fluorescamine staining. The 100-fold diluted supernatant was mixed with a 10 vol % fluorescamine solution (0.5 gL⁻¹ in DMSO) at room temperature and incubated for 15 minutes. The fluorescence intensity of the mixture was measured with a fluorescence spectrometer (excitation wavelength: 395 nm, emission wavelength: 495 nm). The binding dissociation constant Kd was determined assuming a Langmuir type binding.

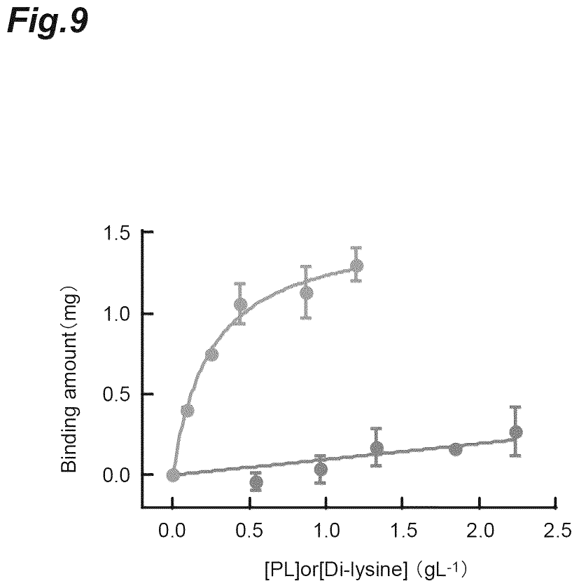

FIG. 9 shows the results obtained by measuring the binding amount (binding amount) of the hydrogel pretreated with AEBSF to PL or di-lysine. In FIG. 9, the results of the binding amount to PL are shown by a light color plot, and the results of the binding amount to di-lysine are shown by a dark color plot.

The AT-gel showed significantly higher affinity for the full-length PL than the di-lysine. The Kds of the AT-gel for PL and di-lysine were 0.25 gL⁻¹ (5 μM) and >2.5 gL^{-b 1} (>9 mM), respectively (FIG. 9). This biased affinity is presumed to be due to a large difference in multivalence between the full-length PL having 370 residues and the di-lysine or tri-lysine.

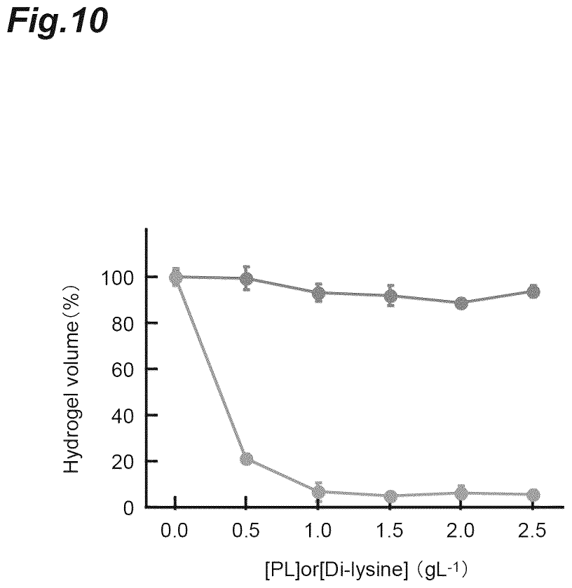

FIG. 10 shows the results obtained by measuring the volume change of the AT-gel pretreated with AEBSF, in response to PL or di-lysine. In FIG. 10, the results of the volume change in response to PL are shown by a light color plot, and the results of the binding amount to di-lysine are shown by a dark color plot.

Although the AEBSF-treated AT-gel shrank in response to the addition of PL, the shrinkage in response to di-lysine was slight (FIG. 10).

### Volume Change of AT-Gel in Response to Addition of Di-Lysine

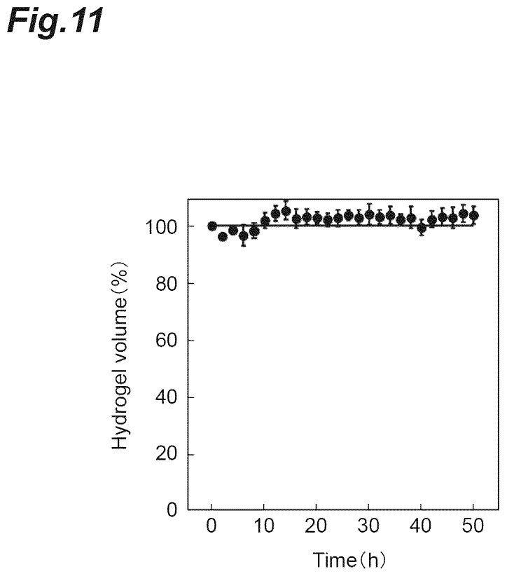

The AT-gel that had been cut into a circle (diameter: 8 mm, thickness: 0.4 mm, polymer weight: 0.5 mg) was immersed in 10 mL of 1 mM NaHCO₃ containing 2 gL⁻¹ di-lysine at 25°° C. The hydrogel volume was examined as a function of time (FIG. 11). It was confirmed that the AT-gel shows no volume change in response to 2 gL⁻¹ di-lysine.

These results led to a conclusion that the re-swelling of the AT-gel is caused by the release of the oligo-lysine hydrolyzed by trypsin due to a decrease in affinity for the hydrogel.

The binding capacity of the AT-gel to PL (3.0±0.17 g/g-polymer) was smaller than that of the supplied PL (4.0 g/g-polymer); however, PL was not present in the supernatant after the cycle. As a result, the transient volume change cycle described above includes a continuous substance exchange between the hydrogel and the bulk solution.

### Contribution of Biased Kinetics Between Anabolic and Catabolic Pathways to Transient Volume Change of Hydrogel

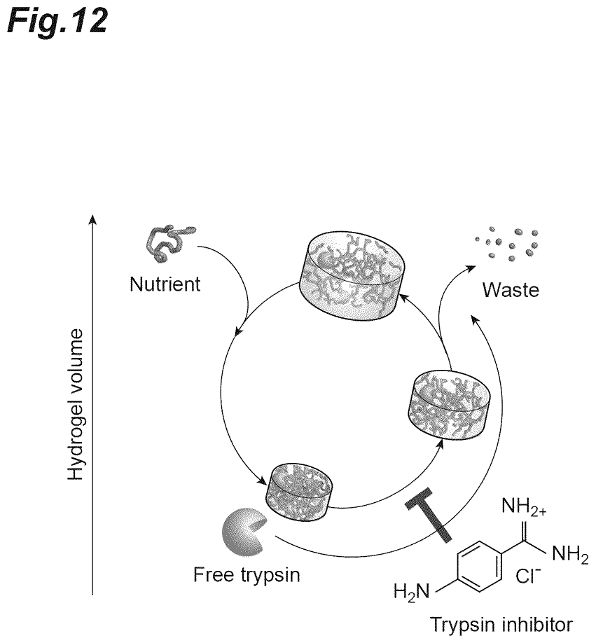

It is considered that in addition to the biased affinity, the transient volume change of the hydrogel through the construction of transient cross-links requires the biased kinetics between the shrinkage (anabolic) pathway and the re-swelling (catabolism pathway. In order to evaluate the influence of the kinetic balance between the construction and destruction of cross-links on the transient volume change, the transient change of the AT-gel due to the addition of PL was investigated in the presence of 4-aminobenzamidine (ABA), a reversible trypsin inhibitor, or free AcTryp in a solution. ABA and free AcTryp were expected to respectively slow down and accelerate the rate of destruction of electrostatic cross-links (FIG. 12).

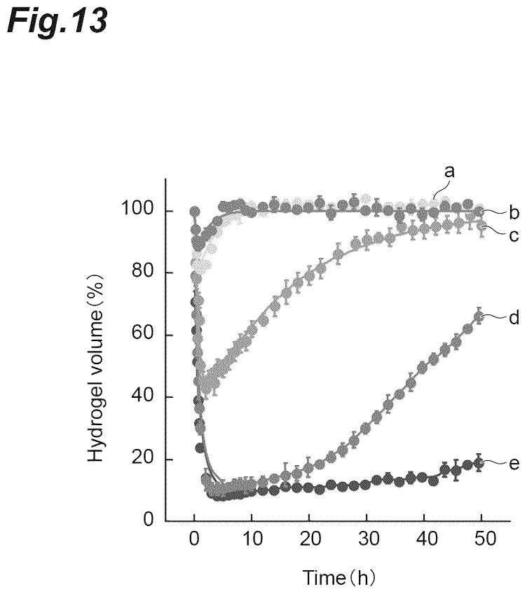

FIG. 13 shows the transient volume change of the AT-gel in the presence or absence (c in the figure) of ABA (1 mM: d in the figure, 5 mM: e in the figure) or free AcTryp (0.1 gL⁻¹: a in the figure, 1.0 gL⁻¹: b in the figure).

In the presence of ABA, the degrees of shrinkage rate and re-swelling rate of the AT-gel increased and reduced, respectively (d and e in FIG. 13). In contrast, in a case where free AcTryp was added to the system, the degree of shrinkage decreased, and rapid re-expansion was observed (a and b in FIG. 13). From these results, it was confirmed that the transient volume change of the AT-gel is caused by the biased kinetics between the fast construction (anabolism) and slow destruction (catabolism of electrostatic cross-links in the polymer network, the electrostatic cross-links being provided by AAc having a density of 17×10³ higher than that of AcTryp. In summary, a conclusion was made that the transient volume change of the AT-gel accompanied by macroscopic and transient volume change is driven by both the biased affinity of PL/oligo-lysine for the hydrogel and the biased kinetics between the rapid construction and slow destruction of electrostatic cross-links.

### Contribution of Stimulus Intensity to Transient Volume Change of Hydrogel

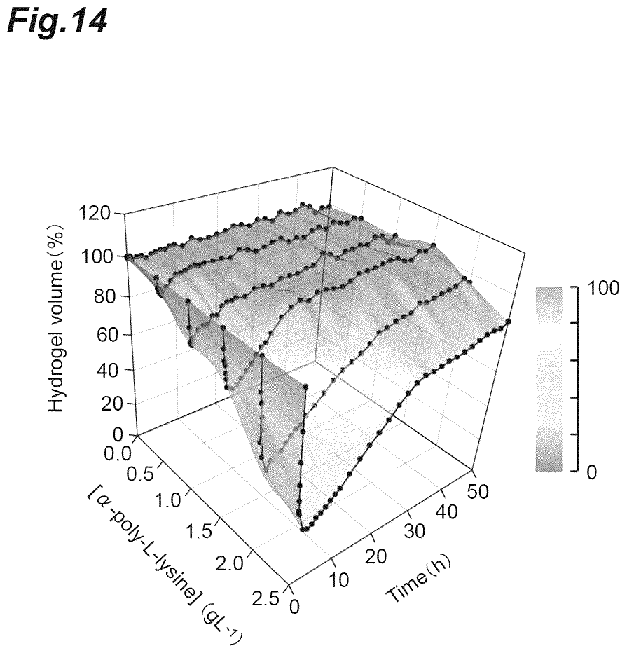

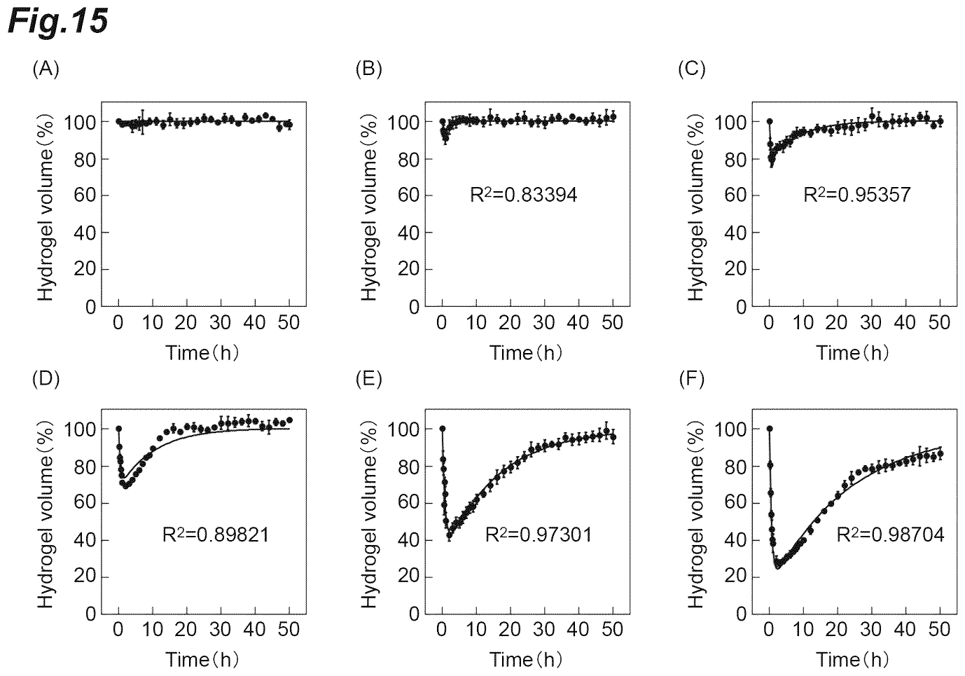

The contribution of the PL concentration to the transient volume change was evaluated (FIG. 14 and FIG. 15). The amplitude of the transient volume change (A), and the time decay constant (Ts) of shrinkage and the time decay constant (Tr) of re-swelling in the transient volume change of the AT-gel were obtained by curve fitting using the double exponential equation (Equation 1) (see the following document d).

Volume (%)=100−A(exp(−t/Ts)−exp(−t/Tr)) (Equation 1)
(d) Clemen, A. E.-M.; Vilfan, M.; Jaud, J.; Zhang, J.; Barmann, M.; Rief. M., Force-Dependent Stepping Kinetics of Myo-sin-V. Biophys. J. 2005, 88, 4402-4410.

The AT-gel that had been cut into a circle (diameter: 8 mm, thickness: 0.4 mm, polymer weight: 0.5 mg) was immersed at 25° C. in 1 mL of 10 mM NaHCO₃ containing PL in various concentrations. The hydrogel volume was examined as a function of time (FIG. 15).

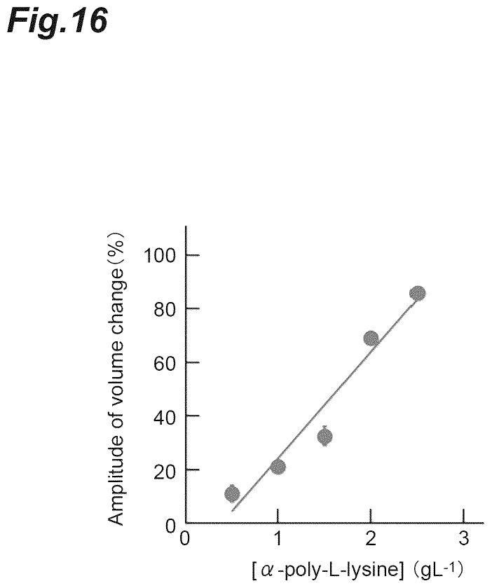

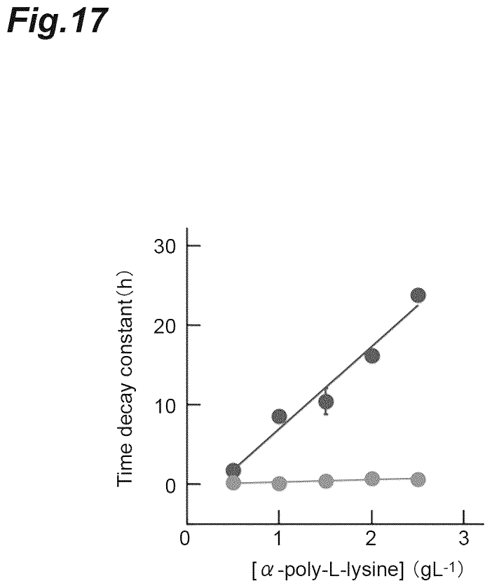

As the PL concentration increased from 0.5 gL⁻¹ to 2.5 gL⁻¹, the amplitude of the volume change of the hydrogel increased from 11%±2.9% to 86%±1.5%. This result indicates that the increase in the number of PLs captured by the hydrogel makes the building blocks for the construction of electrostatic cross-links more abundant, thereby promoting the shrinkage pathway (FIG. 16). FIG. 17 shows the time decay constant with respect to the PLL concentration, where the dark color plot in FIG. 17 shows Tr, and the light color plot in FIG. 17 shows Ts. As shown in FIG. 17, Ts is smaller than Tr under all experimental conditions (FIG. 17). This is consistent with the above discussion. The increase in PL concentration had a slight effect on Ts; however, Tr increased noticeably. As the amount of PL increases, the consumption time becomes longer, and the re-expansion is slowed down. These results led to a conclusion that the amount of PL supplied, that is, the stimulus intensity regulates the degree of the transient shrinkage and the lifetime of the hydrogel by changing the balance between the shrinkage pathway and the re-expansion pathway.

### Secretion of Supported Substance (Payload) Due to Transient Volume Change of Hydrogel

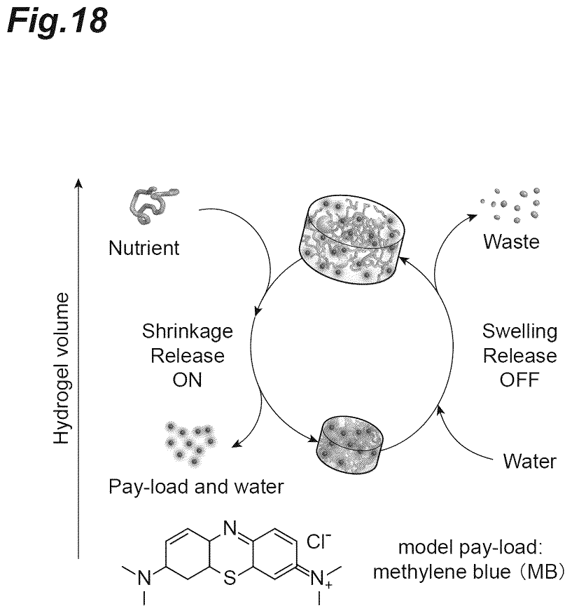

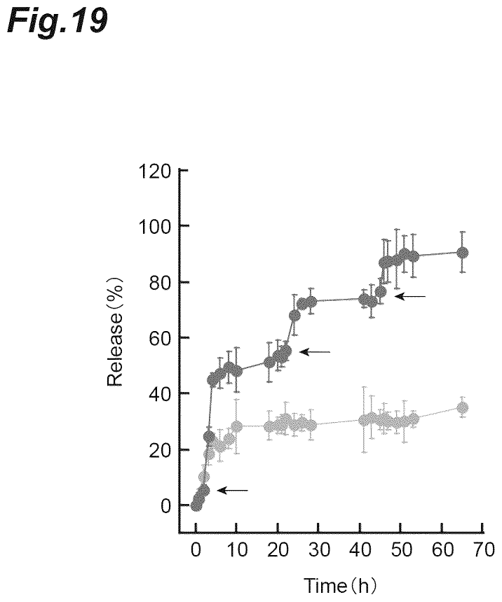

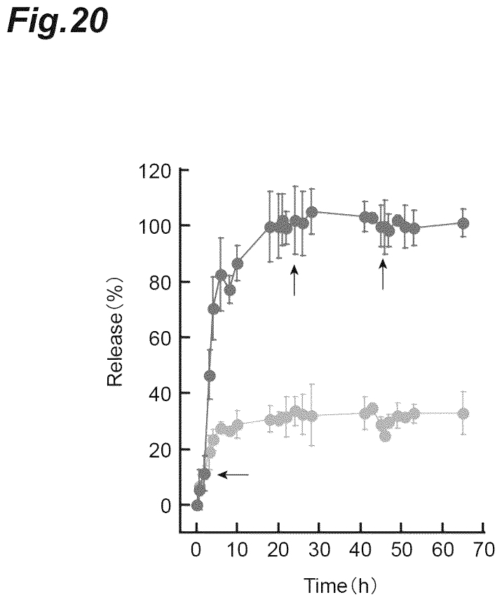

The ability of the AT-gel to convert a primary volume change into an external work was verified. Methylene blue (MB) was used as the model supported substance (Cwalinski, T. et al., J. Clin. Med. 2020, 9, 3538.) to demonstrate a transient secretion of target responsiveness (FIG. 18, FIG. 19, and FIG. 20). The influence of the transient volume change on the release characteristics of the supported substance was revealed by comparing the release of MB from the AT-gel and the A-gel.

The AT-gel and the A-gel, which had been cut into a circle (diameter: 8 mm, thickness: 0.4 mm, polymer weight: 0.5 mg), were immersed in 1 mL of 10 mM NaHCO₃ containing 0.1 mg/mL methylene blue (MB) and incubated at 4° C. for 15 hours. The AT-gel and the A-gel, which had supported MB, were immersed in 1 mL of 10 mM NaHCO₃, and the released MB was quantified by fluorescence spectroscopy (the excitation wavelength and the fluorescence wavelength were λ=633 nm and λ=680 nm, respectively).

In FIG. 19 and FIG. 20, the dark color plot shows the results in a case where PL has been added, and the light color plot shows the results in a case where PL has not been added (spontaneous MB release). Both the AT-gel and the A-gel released MB by adding 1 gL⁻¹ PL (FIG. 19 and FIG. 20). This results indicate that the shrinkage of the hydrogel pushes the water out to release MB (Kikuchi, A. et al., Adv. Drug Delivery Rev. 2002, 54, 53-77.).

The release of MB from the AT-gel reached a plateau at 50%±5.7% within 6 hours (FIG. 19). On the other hand, the A-gel released MB gradually and continuously, and an almost complete release (100%÷11.5%) was observed after 20 hours (FIG. 20). These results indicate that the autonomous release off from the AT-gel has been achieved by the autonomous re-expansion of the hydrogel, which reverses the flow of water (FIG. 18). The release of MB from the AT-gel in association with time change could be stepwise extended over at least three times in response to the addition of 1 gL⁻¹ PL (FIG. 19). A conclusion was made that the AT-gel regulates the secretion of MB in association with time change in response to PL. These results clearly revealed the characteristic function of the AT-gel as compared with a hydrogel (A-gel) without hydrolytic activity and showed potential for a hydrogel that exhibits a transient volume change in response to a biopolymer as an intelligent drug release system.

### CONCLUSION

By being inspired by the processes of nutrient intake, anabolism, catabolismand waste excretion in biological systems, the inventors of the present invention shown that a macroscopic and transient volume change of the hydrogel, which is triggered by a biopolymer, can be realized by combining two functions of affinity and hydrolytic activity of a target biopolymer.

The system proposed in the present invention includes the following steps: (i) intake (a hydrogel takes in a nutrient (PL)), (ii) anabolism (anabolism that constructs electrostatic cross-links as a transient structure), (iii) catabolism the transient structure is destroyed by enzymatic decomposition), and (iv) excretion (hydrolyzed oligo-lysine is autonomously released).

Transient volume change was driven by both the biased affinity of the nutrient and metabolic waste for the hydrogel and the biased kinetics between the fast anabolic pathway and the slow catabolic pathway. The amplitude and rate of the transient volume change were regulated by the nutrient concentration, that is, the stimulus intensity. The hydrogel realized the transient secretion of the supported substance in response to nutrients.

The inventors of the present invention have made it possible to induce a transient volume change by manipulating affinity for a particular target. In the cycle of the transient volume change, the structure of the hydrogel changes macroscopically and temporarily. The present invention is considered to be useful in strategies for designing a drug delivery/release system that responds to a biologically and therapeutically important target and an artificial material in an out-of-equilibrium system for the regulation of a target biological process such as a tissue engineering scaffold.

1. A method for changing a hydrogel volume, the method comprising:
a step A of bringing an ionic polymer into contact with a hydrogel, wherein the hydrogel comprises a site having decomposition activity for the ionic polymer and a cross-linkable functional group that is capable of cross-linking with the ionic polymer through electrostatic interaction, to allow the cross-linkable functional group to cross-link with the ionic polymer, and reducing a volume of the hydrogel; and
a step B of decomposing the ionic polymer cross-linking with the hydrogel by using the site having decomposition activity and discharging at least part of a decomposition product of the ionic polymer from the hydrogel to increase the hydrogel volume.

2. The method according to claim 1, wherein the ionic polymer is an ionic biopolymer.

3. The method according to claim 1, wherein the ionic polymer is a cationic polypeptide having a lysine residue.

4. The method according to claim 1, wherein a molecular weight of the ionic polymer is 530.70 or more.

5. The method according to claim 1, wherein the cross-linkable functional group is at least one selected from the group consisting of a carboxy group and a sulfate group.

6. The method according to claim 1, wherein the volume of the hydrogel is reduced by 10% to 80% by formation of a cross-linking structure with the ionic polymer.

7. A hydrogel comprising:
a site having decomposition activity for an ionic polymer; and
a cross-linkable functional group that is capable of cross-linking with the ionic polymer through electrostatic interaction.

8. The hydrogel according to claim 7, wherein the ionic polymer is a cationic polypeptide having a lysine residue.

9. The hydrogel according to claim 7, wherein a molecular weight of the ionic polymer is 530.70 or more.

10. The hydrogel according to claim 7, wherein the cross-linkable functional group is at least one selected from the group consisting of a carboxy group and a sulfate group.

11. The hydrogel according to claim 7, wherein a volume of the hydrogel is reduced by 10% to 80% by formation of a cross-linking structure with the ionic polymer.
